(19) United States
(12) Patent Application Publication
(10) Pub. No.: US 2022/0333071 A1
(43) Pub. Date: Oct. 20, 2022

(54) METHODS OF MAKING, EXPANDING AND PURIFYING MIDBRAIN DOPAMINERGIC PROGENITOR CELLS

(71) Applicant: BrainXell, Inc., Madison, WI (US)

(72) Inventor: Zhong-Wei DU, Madison, WI (US)

(21) Appl. No.: 17/723,026

(22) Filed: Apr. 18, 2022

Related U.S. Application Data
(60) Provisional application No. 63/176,006, filed on Apr. 16, 2021.

(51) Int. Cl. C12N 5/079

(52) U.S. Cl. CPC C12N 5/0622; C12N 2501/415; C12N 2501/119; C12N 2501/999; C12N 2506/45

(57) ABSTRACT

The present invention provides methods of producing, purifying and expanding mDA progenitor cells.

### CROSS REFERENCE TO RELATED APPLICATIONS

This application claims the benefit of priority to U.S. Provisional Patent Application Ser. No. 63/176,006 filed on Apr. 16, 2021 the contents of which are hereby incorporated by reference in its entirety.

### FIELD OF THE INVENTION

The present invention relates generally to methods of producing, purifying and expanding midbrain dopaminergic progenitor cells.

### BACKGROUND OF THE INVENTION

Cell populations that retain the ability to differentiate into numerous specialized cell types are useful for developing large numbers of lineage specific differentiated cell populations. These cell populations that retain a capability for further differentiation into specialized cells contain pluripotent cells. Pluripotent cells may be from embryonic and/or nonembryonic somatic stem cell origin. These lineage specific differentiated cell populations are contemplated to find use in cell replacement therapies for patients with diseases resulting in a loss of function of a defined cell population.

In addition to their direct therapeutic value, lineage specific differentiated cells are also valuable research tools for a variety of purposes including in vitro screening assays to identify, confirm, and test for specification of function or for testing delivery of therapeutic molecules to treat cell lineage specific disease.

Previously embryonic and somatic stem cells were used as therapeutics and model systems for neurodegenerative diseases. Research and technological developments relating to directed differentiation of embryonic and somatic stem cells has taken place in the field of diseases of the central nervous system (CNS), such as for Huntington's, Alzheimer's, Parkinson's, and multiple sclerosis.

Therefore, there is a need for methods to obtain cell populations capable of being used as a therapeutic for treating neurodegenerative diseases.

### SUMMARY OF THE INVENTION

In some aspects, the disclosure provides a method of purifying a population of midbrain dopamine (mDA) progenitor cells by isolating CD166 expressing cells from a differentiated population of progenitor cells to produce a purified population of mDA progenitor cells.

In some embodiments, the differentiated population of progenitor cells is derived from pluripotent stem cells. In some embodiments, the progenitor cells are induced pluripotent stem cells (iPSC). In some embodiments, the differentiated population of progenitor cells is derived from embryonic stem cells (ESC). In some embodiments, the differentiated population of progenitor cells is derived from midbrain floor plate progenitor cells. In some embodiments, the differentiated population of progenitor cells comprise cells expressing one or more genes selected from EN1, PAX8, OTX2, LMX1A, FOXA2, Conn, and CD166.

In some embodiments, the differentiated population of progenitor cells comprise less than 70% CD166+ cells. In some embodiments, the differentiated population of progenitor cells comprise less than 70% CD166+, Corin+ double positive cells. In some embodiments, the purified population of mDA progenitor cells comprise at least 70% CD166+ cells.

In some embodiments, the methods described herein further comprise expanding the purified population of mDA progenitor cells. In some embodiments, expanding the cell population comprises contacting the purified population of mDA progenitor cells with, a SHH agonist, a BMP inhibitor, a Nodal/Activin inhibitor, a GSK3 inhibitor and a PORCN inhibitor for period of time until a sufficient number of mDA progenitor cells are produced. In some embodiments, the expansion does not alter the phenotype of the purified population of mDA cells.

In some aspects, the disclosure provides a method of expanding a population of midbrain (mDA) cells, wherein the method does not alter the phenotype of the mDA cells, comprising providing a population of mDA cells and contacting the culture with SHH agonist, a BMP inhibitor, a Nodal/Activin inhibitor, a GSK3 inhibitor and a PORCN inhibitor for period of time until a sufficient number of mDA progenitor cells are produced. In some embodiments, prior to expansion the population of mDA cells are at least 60, 70, 80, 90% CD166+.

In some aspects, the disclosure provides a method of expanding a population of midbrain (mDA) cells, wherein the method does not alter the phenotype of the mDA cells comprising: a) isolating CD166 expressing cells from the population mDA cells to provide a purified population of mDA cells; and b) culturing the purified population of mDA cells in the presence of, a SHH agonist, a BMP inhibitor, a Nodal/Activin inhibitor, a GSK3 inhibitor and a PORCN inhibitor for period of time until a sufficient number of mDA progenitor cells are produced thereby expanding the population of mDA cells. In some embodiments, the purified population of mDA progenitor cells comprise at least 60, 70, 80, or 90% CD166+ cells. In some embodiments, the CD166 expressing cells are isolated by FACS or MACS.

In some aspects, the disclosure provides a method of expanding a population of midbrain (mDA) cells, wherein the method does not alter the phenotype of the mDA cells comprising: a) providing a population of at least 70% CD166+ mDA cells; and b) culturing the CD166+ mDA cells in the presence of a SHH agonist, a BMP inhibitor, a Nodal/Activin inhibitor, a GSK3 inhibitor and a PORCN inhibitor for period of time until a sufficient number of mDA progenitor cells are produced thereby expanding the population of mDA cells. In some embodiments, the period of time is about between 1-5 weeks. For example, 1, 2, 3, 4, 5 or more weeks. In some embodiments, the sufficient number of mDA progenitor cells is at least 10⁹ cells.

In some embodiments, the BMP inhibitor is DMH1. In some embodiments, the concentration of DMH1 is between about 0.1 μM to about 10 μM. In some embodiments, the concentration of DMH1 is between about 1 μM to about 5 μM. In some embodiments, the concentration of DMH1 is about 2 μM.

In some embodiments, the GSK3 inhibitor is CHIR99021. In some embodiments, the concentration of CHIR99021 is between about 0.1 μM and about 5 μM. In some embodiments, the concentration of CHIR99021 is between about 1 μM and about 5 μM. In some embodiments, the concentration of CHIR99021 is about 3 μM.

In some embodiments, the SHH agonist is SAG. In some embodiments, the concentration of SAG is between about 0.02 μM and about 5 μM. In some embodiments, the concentration of SAG is between about 0.1 μM and 2 μM. In some embodiments, the concentration of SAG is about 1 μM.

In some embodiments, the Nodal/Activin inhibitor is SB431542. In some embodiments, the concentration of DMH1 is between about 0.1 μM to about 10 μM. In some embodiments, the SB431542 is at a concentration of between about 1 μM and about 5 μM. In some embodiments, the SB431542 is at a concentration of about 2 μM.

In some embodiments, the PORCN inhibitor is Wnt-059. In some embodiments, the concentration of Wnt-059 is between about 0.1 μM and about 5 μM. In some embodiments, the concentration of Wnt-059 is about 0.5 μM.

In some aspects, the disclosure provides an in vitro method of producing a midbrain dopamine (mDA) progenitor cell population comprising: a) culturing a population of less than 5 million stem cells for two (2) consecutive days such that small pluripotent stem cell clusters are formed to produce a first cell population; b) contacting the first cell culture with the BMP inhibitor, the GSK3 inhibitor, the SHH agonist, and Nodal/Activin inhibitor for about 7-12 consecutive days to produce a first midbrain floor plate progenitor cell population; c) passaging the midbrain floor plate progenitor cell population to produce a passaged cell population; d) contacting the passaged cell population with the GSK3 inhibitor and the SHH agonist for about two (2) days to produce a second midbrain floor plate progenitor cell population; e) contacting the second midbrain floor plate progenitor cell population with FGF8b and the SHH agonist for about six (6) consecutive days thereby producing a mDA progenitor population; f) contacting the mDA progenitor cell culture with the SHH agonist, the BMP inhibitor, the Nodal/Activin inhibitor, the GSK3 inhibitor and a PORCN inhibitor for about two (2) days to produce an expanded mDA progenitor cell population; g) purifying the mDA progenitor cell population by sorting CD166 expressing cells from the cell population to produce a purified mDA progenitor cell population; and h) expanding the purified mDA progenitor cell population by contacting the culture with, a SHH agonist, a BMP inhibitor, a Nodal/Activin inhibitor, a GSK3 inhibitor and a PORCN inhibitor for a second period of time until a sufficient number of mDA progenitor cells are produced.

In some embodiments, the first period of time is about 2 days. In some embodiments, the second period of time is between about 7-12 days.

In some embodiments, the population of stem cells is less than 1 million cells. In some embodiments, the population of stem cells is less than 500,000 cells.

In some embodiments, the sufficient number of mDA progenitor cells is at least 10⁶ cells. In some embodiments, the sufficient number of mDA progenitor cells is at least 10⁷ cells. In some embodiments, the sufficient number of mDA progenitor cells is at least 10⁸ cells.

In some embodiments, the BMP inhibitor is DMH1. In some embodiments, the concentration of DMH1 is between about 0.1 μM to about 10 μM. In some embodiments, the concentration of DMH1 is between about 1 μM to about 5 μM. In some embodiments, the concentration of DMH1 is about 2 μM.

In some embodiments, the GSK3 inhibitor is CHIR99021. In some embodiments, the concentration of CHIR99021 is between about 0.1 μM and about 5 μM. In some embodiments, the GSK3 inhibitor is CHIR99021 and the concentration of CHIR99021 in step (b) is between about 0.7 μM and about 1.2 μM.

In some embodiments, the concentration of CHIR99021 in step (d) is between about 0.1 μM and about 5 μM. In some embodiments, the concentration of CHIR99021 is 3 μM.

In some embodiments, the GSK3 inhibitor is CHIR99021 and the concentration of CHIR99021 in step (0 and (h) is between about 1.0 μM and about 5 μM. In some embodiments, the concentration of CHIR99021 is about 3 μM.

In some embodiments, the SHH agonist is SAG. In some embodiments, SAG is at a concentration of between about 0.02 μM and 5 μM. In some embodiments, the SHH agonist is SAG and the concentration of SAG in step (b) is between about 0.1 μM and 2 μM. In some embodiments, the concentration of SAG is about 1 μM.

In some embodiments, the SHH agonist is SAG and the concentration of SAG in step (d), (f) and (h) is between about 0.02 μM and about 1 μM. In some embodiments, SAG is at a concentration of between about 0.05 μM and about 0.5 μM. In some embodiments, SAG is at a concentration of about 0.1 μM.

In some embodiments, the Nodal/Activin inhibitor is SB431542. In some embodiments, the concentration of DMH1 is between about 0.1 μM to about 10 μM. In some embodiments, the SB431542 is at a concentration of between about 0.5 μM and about 5 μM. In some embodiments, the SB431542 is at a concentration of about 2 μM.

In some embodiments, the PORCN inhibitor is a porcupine (PORCN) inhibitor. In some embodiments, the PORCN inhibitor is Wnt-059. In some embodiments, the concentration of Wnt-059 is between about 0.1 μM and about 1 μM. In some embodiments, the concentration of Wnt-C9 is about 0.5 μM.

In some embodiments, the FGF8b is at a concentration of between about 5 ng/ml and about 50 ng/ml. In some embodiments, the FGF8b in step (e) is at a concentration of about 20 ng/ml.

In some embodiments, the CD166 expressing cells are isolated by FACS or MACS. Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a flow chart depicting differentiation of midbrain dopaminergic (Dopa) progenitors from pluripotent stem cells (PSC), sorting pure Dopa progenitors with CD166 antibody if needed, and expansion of these Dopa progenitors under specified conditions. Abbreviations: PSC (pluripotent stem cell); Dopa (dopaminergic).

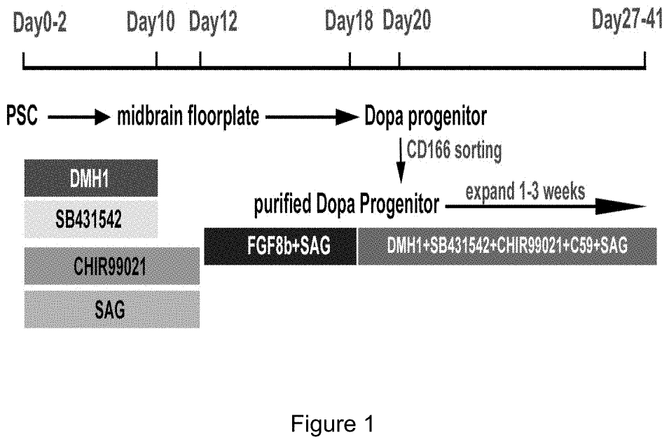

FIG. 2 discloses the expansion of pure population of midbrain Dopa progenitors. (A) compare different expansion conditions with different dose of CHIR99021 and Wnt-059, the condition #3 with 3 μM CHIR99021 and 0.5 μM Wnt-059 showed the best condition to maintain CD166+/Corin+ pure Dopa progenitors. (B) after expanded for 3 passages in 3 weeks, TH-mCherry reporter ESC derived Dopa progenitors was maintained at 79.62% CD166+/Corin+ by FACS analysis, and showed 30.66% TH-mCherry expression after neuronal differentiation (C).

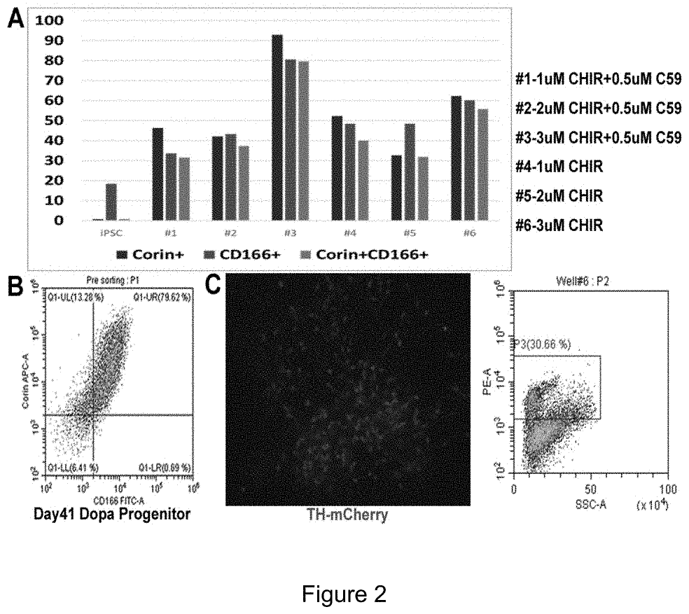

FIG. 3 showed MACS sorting of iPSC derived midbrain Dopa progenitors. The CD166+ purity increased from 39.12% before sorting into 87.09% after sorting.

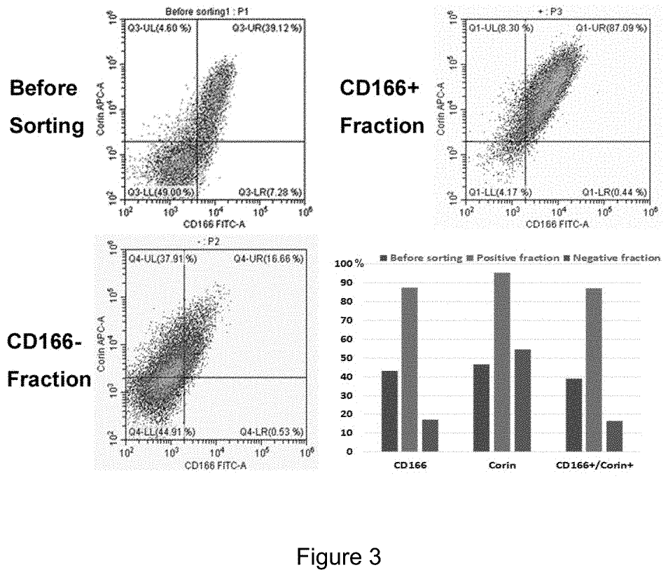

FIG. 4 shows the expansion of sorted iPSC derived midbrain Dopa progenitors. (A) Bright field image showed the morphology of expanding Dopa progenitors. (B) Cell numbers increased from 50 million into 5.8 billion after 5 passages. (C) After neuronal differentiation, TH+ neurons maintained at around 30% during expansion.

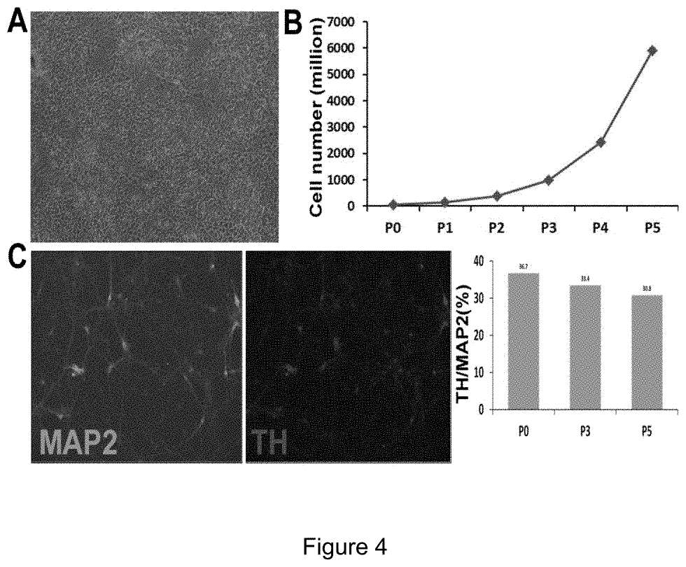

### DETAILED DESCRIPTION OF THE INVENTION

The invention is based in part upon the discovery that midbrain dopaminergic (mDA) progenitor cells can be expanded extensively in vitro without altering the phenotype of the cells. Further, included by the invention are methods of purifying mDA progenitor cells. Also included in the inventions are methods of producing large numbers of mDA precursor cells from less than 5 million stem cells.

Existing methods of producing mDA progenitor cells, are limited by the starting number of pluripotent stem cells utilized. In contrast, the methods described herein are not limited by the starting population of pluripotent stem cell and therefore allows the production of over 1 billion mDA progenitor cells from less than 5 million stem cells.

Accordingly, the methods described herein result in (1) an enriched population of mDA progenitors and resultant neurons; (2) less contaminating pluripotent cells; (3) less contaminating non mDA progenitors and (4) a more reproducible and defined final product.

The present disclosure provides for in vitro methods for producing, purifying and expanding mDA progenitor cells.

The mDA progenitor cells are produced from stem cells using methods known in the art or the methods described herein. For example, pluripotent stem cells can be differentiated into midbrain dopaminergic (mDA) progenitors by directing cells through a midbrain floor plate progenitor stage. Stem cells that can be used in accordance with the methods described herein include human and nonhuman primate or rodent stem cells, and engineered (genetically or otherwise), pluripotent cells.

Human stem cells include for example, human embryonic stem cells (hESC), human pluripotent stem cells (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In other embodiments, the human stem cell is a human induced pluripotent stem cell (hiPSC).

Midbrain dopaminergic (mDA) progenitor cells are expanded by contacting a substantially pure population of mDA progenitor cells with an effective amount of, a Sonic Hedge Hog (SHH) agonist, a bone morphogenetic protein (BMP) inhibitor, a Nodal/Activin inhibitor, a glycogen synthase kinase 3 (GSK-3) inhibitor and a Porcupine (PORCN) inhibitor for period of time until a sufficient number of mDA progenitor cells are produced.

In certain aspects, the mDA progenitor cells are purified prior to the expansion step. Optionally, the expanded mDA progenitor cells may be purified and then expanded for a second time. The procedure of purifying and expanding can occur 2, 3, 4, 5 or more times, until the desired number of mDA progenitor cells is achieved.

The disclosure also provides methods of purifying mDA progenitors from a population of cells by selecting CD166 expressing cells. Methods of selecting for CD166 expressing cells are known in the art and include affinity chromatography techniques such as using CD166 coated magnetic beads. The population of cells are cells that have undergone the differentiation into mDA progenitor cells or are a population of cells known to contain mDA progenitor cells. For example, the population of cells were derived from pluripotent stem cells (e.g., iPSC or ESC) that were differentiated into midbrain dopaminergic (mDA) progenitors by directing cells through a midbrain floor plate progenitor stage. Purification in accordance with the methods disclosed herein achieves a population of mDA progenitor cells having a purity at least 70%, 75%, 80%, 85%, 90%, 95% or more.

Populations of mDA progenitor cells are identified by methods known in the art. For example, mDA progenitor cells can be identified by positive or negative selection of markers indicative of mDA progenitor cells. Positive mDA progenitor cell markers include one or more of EN1, PAX8, OTX2, LMX1A, FOXA2, Corin, and CD166. Negative mDA progenitor cell markers include one or more of iPSC-Oct4, LIN28, Forebrain-NKX2.1, BARHL1, Hindbrain-HOXA2, NKX2.2, Serotonin progenitor-FEV and GATA2. Positive or negative mDA progenitor cell markers can be measured by methods known in the art such as quantitative PCR or immunostaining.

A substantially pure population of mDA progenitor cells is a population of mDA progenitor cells where at least 70%, 75%, 80%, 85%, 90%, 95% or more for the cells express CD166 on their cell surface. More specifically, at least 70%, 75%, 80%, 85%, 90%, 95% or more for the cells express CD166 and Corin on their cell surface. Cell surface expression of CD166 and Corin can be determined by FACS.

The methods further include contacting cells with one or more activator of SHH signaling. As used herein, the term “Sonic hedgehog,” “SHH,” or “Shh” refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). Shh typically binds to PTC which then allows the activation of SMO as a signal transducer. In the absence of SHH, PTC typically inhibits SMO, which in turn activates a transcriptional repressor so transcription of certain genes does not occur. When Shh is present and binds to PTC, PTC cannot interfere with the functioning of SMO. With SMO uninhibited, certain proteins are able to enter the nucleus and act as transcription factors allowing certain genes to be activated (see, Gilbert, 2000 Developmental Biology (Sunderland, Mass., Sinauer Associates, Inc., Publishers).

In certain embodiments, an activator of Sonic hedgehog (SHH) signaling refers to any molecule or compound that activates a SHH signaling pathway, including a molecule or compound that binds to PTC or a Smoothened agonist and the like.

Examples of such compounds are recombinant SHH, purified SHH, a protein Sonic hedgehog (SHH) C25II (i.e., a recombinant N-Terminal fragment of a full-length murine sonic hedgehog protein capable of binding to the SHH receptor for activating SHH, for example, R and D Systems catalog number: 464-5H-025/CF) and a small molecule Smoothened agonist such as, for example, SAG, derivatives thereof, and mixtures thereof “SAG” refers to a molecule with a number CAS 912545-86-9 and 364590-63-6 (hydrocloride) and the name 3-chloro-N-[(1r,40-4-(methylamino)cyclohexyl]-N-[3-(pyridin-4-yl)benzyl]benzo[b]thiophene-2-carboxamide.

In specific embodiments, the SHH agonist is SAG and is at a concentration of between about 0.01 and 10 μM. Preferably, the concentration of SAG is about 0.1 μM.

In certain embodiments the inhibitor of BMP signaling, results in inhibition of SMAD signaling. In other embodiments the inhibitor of BMP signaling results in the selective inhibition of BMP.

Non-limiting examples of inhibitors of BMP signaling are disclosed in WO2011/149762, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20, which are incorporated by reference in their entireties. In certain embodiments, the inhibitor of BMP signaling is the small molecule DMH-1, derivatives thereof, and mixtures thereof “DMH1” refers to a molecule with a number CAS 1206711-16-1-41-9, and a name of 4-[6-[4-(1-Methylethoxy)phenyl]pyrazolo[1,5-a]pyrimidin-3-yl]-quinoline.

DMH-1 is a selective inhibitor of bone morphogenic protein (BMP) type-I receptor activin receptor-like kinase 2 (ALK2) receptor in in vitro kinase assays). Exhibits 6- and 19-fold selectivity for ALK-2 over ALK-1 and ALK-3, respectively, and no significant inhibition of AMPK, ALK5, KDR (VEGFR-2) or PDGFRβ receptors. Blocks BMP4-induced phosphorylation of Smads 1, 5 and 8 in HEK293 cells. (Neely et al. ACS Chem. Neurosci., 2012; 3:482, which are incorporated by reference in its entirety)

In specific embodiments, the BMP inhibitor is DMH1 and is at a concentration of between about 0.1 and 10 μM, or between about 1 and 5 μM, or between about 1.5 and 3.0 μM. Preferably, the concentration of DWH1 is about 2 μM.

In certain embodiments, the inhibitor of Nodal/Activin signaling neutralizes the ligands including TGFβs, bone morphogenetic proteins (BMPs), Nodal, and activins, or blocking their signal pathways through blocking the receptors and downstream effectors. Non-limiting examples of inhibitors of Nodal-Activin signaling are disclosed in WO/2010/096496, WO/2011/149762, WO/2013/067362, WO/2014/176606, WO/2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), which are incorporated by reference in their entireties herein for all purposes.

In certain embodiments, the one or more inhibitor of Nodal-Activin signaling is the small molecule SB431542, derivatives thereof, and mixtures thereof “SB431542” refers to a molecule with a number CAS 301836-41-9, and a name of 4-[4-(1,3-benzodioxol-5-yl)-5-(2-pyridinyl)-1H-imidazol-2-yl]-benzamide.

In specific embodiments, the Nodal/Activin inhibitor is SB431542 and is at a concentration of between about 0.1 and 10 μM, or between about 1 and 5 μM, or between about 1.5 and 3.0 μM. Preferably, the concentration of SB431542 is about 2 μM.

In certain embodiments, cells are contacted with one or more inhibitor of GSK-3. As used herein, the term “GSK-3” or “Glycogen synthase kinase 3” in reference to a serine/threonine protein kinase that phosphorylate either threonine or serine, and this phosphorylation controls a variety of biological activities, such as glycogen metabolism, cell signaling, cellular transport, and others.

In certain embodiments, GSK-3 is also integrally tied to pathways of cell proliferation. GSK-3 has been shown to phosphorylate β-catenin, thus targeting it for degradation. Thus, GSK-3 is a part of the canonical Beta-catenin/Wnt pathway, which signals the cell to divide and proliferate. A GSK3β inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 3β inhibitor” refers to a compound that inhibits a glycogen synthase kinase 3β enzyme, for example, see, Doble, et al., J Cell Sci. 2003; 116:1175-1186, which is incorporated by reference herein in its entirety.

As used herein, the term “WNT” or “wingless” in reference to a signaling pathway refers to a signal pathway composed of Wnt family ligands and Wnt family receptors, such as Frizzled and LRPDerailed/RYK receptors, mediated with or without β-catenin. For the purposes described herein, a preferred WNT signaling pathway includes mediation by β-catenin, e.g., WNT/-catenin.

Non-limiting examples of GSK3β inhibitors are disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, which are incorporated by reference in their entireties. In certain embodiments, the GSK3β inhibitor is the small molecule CHIR99021, derivatives thereof, and mixtures thereof “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl) pyrimidin-2-ylamino) ethylamino) nicotinonitrile.

CHIR99021 is highly selective, showing nearly thousand-fold selectivity against a panel of related and unrelated kinases, with an IC50=6.7 nM against human GSK3-β and nanomolar IC50 values against rodent GSK3-β homologs.

In specific embodiments, the GSK3β inhibitor is CHIR99021 and is at a concentration of between about 0.1 and 10 μM, or between about 1 and 5 μM, or between about 1.5 and 3.0 μM. Preferably, the concentration of CHIR99021 is about 3 μM.

The methods further include contacting cells with one or more inhibitors of PORCN. Porcupine (PORCN) is an O-acyltransferase that catalyzes the palmitoylation of Wnt proteins, a post-translational modification that is necessary for their secretion and activity. In certain embodiments, an inhibitor of PORCN refers to any molecule or compound that inhibits a WNT signaling pathway.

The rate of Wnt secretion is one potential control point for Wnt signaling. Whereas there are 19 distinct Wnt genes and multiple Wnt receptors, there appears to be a single conserved and essential Wnt biogenesis pathway. The first step in Wnt protein production is translation in the endoplasmic reticulum, followed by post-translational modification by the ER-resident enzyme PORCN, PORCN is a membrane-bound O-acyltransferase (MBOAT), and it catalyzes the palmitoylation of the serine corresponding to Ser-209 of WNT3A. This modification is absolutely required for the next step in Wnt secretion, binding to the carrier protein WLS. In addition, palmitoylation is essential for the ability of Wnts to interact with Frizzled receptors at the cell surface. Small molecule inhibitors of PORCN have been developed that inhibit Wnt signaling.

Examples of such compounds includes for example, the small molecule Wnt-059 derivatives thereof, and mixtures thereof “Wnt-059” refers to a molecule with a number CAS 1243243-89-1 and the name 4-(2-Methyl-4-pyridinyl)-N-[4-(3-pyridinyl) phenyl]benzeneacetamide. Wnt-059 is a potent PORCN inhibitor. Wnt-059 inhibits porcupine (PORCN) required for Wnt palmitoylation, secretion, and biological activity. Inhibits Wnt-mediated transcription (IC50=74 pM) and cell proliferation.

In specific embodiments, the PORCN inhibitor is Wnt-059 and is at a concentration of between about 0.1 and 5 μM, or between about 0.2 and 4 μM, or between about 0.3 and 3.0 μM or between about 0.4 and 2.0 μM, or between about 0.5 and 1.0 μM. Preferably, the concentration of Wnt-059 is about 0.5 μM.

In certain embodiments, the above-described inhibitors and activators are added to a cell culture medium comprising the stem cells or mDA progenitor cells. Suitable cell culture media include, but are not limited to, DMEM/F12 medium, Neurobasal medium (NB), N2 medium, B-27 medium, and Essential 8/Essential 6. (“E8/E6”) medium, Essential 8 (“E8’) and combinations thereof. All of these mediums are commercially available.

In certain embodiments, the cell culture medium is an E8 medium. E8 medium is feeder-free, animal component-free culture medium for human embryonic stem (ES) cells and human induced pluripotent stem (iPS) cells. It is based on the E8 formulation developed by the laboratory of Dr. James Thomson (University of Wisconsin-Madison), In certain embodiments, an E8 cell culture medium comprises recombinant laminin-511 (iMatrix-511).

In certain embodiments, mDA cells are contacted with a SHH agonist, a BMP inhibitor, a Nodal/Activin inhibitor, a GSK3 inhibitor and a PORCN inhibitor.

In certain embodiments, the disclosure provides for in vitro methods for inducing differentiation of human stem cells into mDA precursors. Specifically, the methods induce differentiation into mDA precursors by utilizing a stem cell population of less than 5 million stem cells, less than 4 million stem cells, less than 3 million stem cells, less than 2 million stem cells, less than 1 million stems cells, less than 500,000 stem cells, less than 500,000 stem cells, less than 400,000 stem cells, less than 300,000 stem cells, less than 200,000 stem cells, less than 100,000 stem cells.

The population of stem cells are cultured for a first period of time until small cell clusters are formed. The stem cells are cultured in any PSC media known in the art, such as those described above. Preferably, the stem cells are cultured in E8 medium with iMatrix-511. The starting cell density is about 5,000 to 50,000 cells/cm². For example, the starting cell density is 20,000 cells/cm². The first period of time is 1, 2, 3, 4, or 5 days. Optimally, the first period of time is 2 days.

The culture of small cell clusters are contacted with the BMP inhibitor, a GSK3 inhibitor, the SHH agonist, and a Nodal/Activin inhibitor for about seven to twelve (7-12) consecutive days to produce a midbrain floor plate progenitor cell population.

The BMP inhibitor is DMH1 and is at a concentration of between about 0.1 and 10 μM, or between about 1 and 5 μM, or between about 1.5 and 3.0 μM. Preferably, the concentration of DMH1 is about 2 μM.

The GSK3 inhibitor is CHIR99021. CHIR99021 is added to the culture at a first (i.e., initial) concentration that is lower than a second concentration. The initial concentration of between about 0.7 and 1.2 μM, The initial concentration of CHIR99021 is about 0.7 μM, or about 0.8 μM, or about 0.9 μM, or about 1.0 μM, or about 1.1 μM and about 1.2 μM. The initial concentration is for a period of about 7-12 days after which the concentration of CHIR99021 is increased to a concentration of between about 0.1 and 10 μM, or between about 1 and 10 μM, or between about 1 and 5 μM. The second concentration of CHIR99021 is about 3 μM.

The SHH agonist is SAG and is at a concentration of between about 0.1 and 10 μM, or between about 0.5 and 5 μM, Preferably, the concentration of SAG is about 1 μM.

The Nodal/Activin inhibitor is SB431542 and is at a concentration of between about 0.1 and 10 μM, or between about 1 and 5 μM, or between about 1.5 and 3.0 μM. Preferably, the concentration of SB431542 is about 2 μM.

The midbrain floor plate progenitor cell population is passaged at 1:2 and contacted with a GSK3 inhibitor and a SHH agonist for about two (2) days followed by the addition of FGF8b and a SHH agonist for about six (6) consecutive days. The GSK3 inhibitor is CHIR99021 and is at a concentration of between about 0.1 and 10 μM, or between about 1 and 5 μM. Preferably, the concentration of CHIR99021 is about 3 μM. The SHH agonist is SAG and is at a concentration of between about 0.05 and 5 μM, or between about 0.01 and 1 μM, or between about 0.1 and 0.5 μM. Preferably, the concentration of SAG is about 0.1 μM. The FGF8b is at a concentration of between about 5-50 μg/mL, or between about 10 and 50 μg/mL, or between about 10 and 40 μg/mL, or between about 15 and 30 μg/mL. Preferably, the concentration of FGF8b is about 20 μg/mL.

The resultant mDA progenitor cell population can be purified and/or expanded by the methods described above.

The mDA precursors produced by the disclosed methods can be used for treating a neurodegenerative disorder by administering an effective amount of the presently disclosed mDA precursors into a subject suffering from a neurodegenerative disorder. The method alleviates a sign or symptom of a neurodegenerative disorder.

Neurodegenerative disorders include Parkinson's disease, Huntington's disease, Alzheimer's disease, and multiple sclerosis.

Primary motor signs of Parkinson's disease include, for example, but not limited to, tremor of the hands, arms, legs, jaw and face, bradykinesia or slowness of movement, rigidity or stiffness of the limbs and trunk and postural instability or impaired balance and coordination.

In certain embodiments, the neurodegenerative disease is a parkinsonism disease, which refers to diseases that are linked to an insufficiency of dopamine in the basal ganglia, which is a part of the brain that controls movement. Symptoms include tremor, bradykinesia (extreme slowness of movement), flexed posture, postural instability, and rigidity. Non-limiting examples of parkinsonism diseases include corticobasal degeneration, Lewy body dementia, multiple system atrophy, and progressive supranuclear palsy.

The mDA precursors can be administered or provided systemically or directly to a subject for treating or preventing a neurodegenerative disorder. In certain embodiments, the mDA precursors are directly injected into an organ of interest (e.g., the central nervous system (CNS) or peripheral nervous system (PNS)). In certain embodiments, the mDA precursors are directly injected into the striatum.

The mDA precursors can be administered in any physiologically acceptable vehicle. Pharmaceutical compositions comprising the mDA precursors and a pharmaceutically acceptable vehicle are also provided. The mDA precursors and the pharmaceutical compositions comprising said cells can be administered via localized injection, orthotopic (OT) injection, systemic injection, intravenous injection, or parenteral administration. In certain embodiments, the mDA precursors are administered to a subject suffering from a neurodegenerative disorder via orthotopic (OT) injection.

The mDA precursors and the pharmaceutical compositions comprising said cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived precursors.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the mDA precursors. This will present no problem to those skilled in chemical and pharmaceutical principles, or problems can be readily avoided by reference to standard texts or by simple experiments (not involving undue experimentation), from this disclosure and the documents cited herein.

In certain non-limiting embodiments, the mDA precursors described herein are comprised in a composition that further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into midbrain DA cells.

One consideration concerning the therapeutic use of the mDA precursors is the quantity of cells necessary to achieve an optimal effect. An optimal effect includes, but is not limited to, repopulation of CNS and/or PNS regions of a subject suffering from a neurodegenerative disorder, and/or improved function of the subject's CNS and/or PNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurodegenerative disorder or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the mDA precursors is an amount that is sufficient to repopulate CNS and/or PNS regions of a subject suffering from a neurodegenerative disorder. In certain embodiments, an effective amount of the mDA precursors is an amount that is sufficient to improve the function of the CNS and/or PNS of a subject suffering from a neurodegenerative disorder, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS and/or PNS.

The quantity of cells to be administered will vary for the subject being treated. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### Definitions

As used herein, the term “inhibitor” in reference to inhibiting a signaling target or a signaling target pathway refers to a compound that interferes with (i.e. reduces or eliminates or suppresses) a resulting target molecule or target compound or target process, such as a particular differentiation outcome, (for example, suppresses an active signaling pathway promoting a default cell type differentiation, thereby inducing differentiation into a non-default cell type) when compared to an untreated cell or a cell treated with a compound that does not inhibit a treated cell or tissue.

As used herein, the term “neural cell” or “neuronal cell” refers to a cell that in vivo would become part of the nervous system and in culture is obtained by methods of the present inventions, for example, CNS progenitor cells, patternable (i.e. a cell capable of undergoing further differentiation) neuronal populations of motorneurons and dopaminergic neurons, placodal precursor cells, high efficiency motor neuron cells, etc.

As used herein, the term “fate” in reference to a cell, such as “cell fate determination” in general refers to a cell with a genetically determined lineage whose progeny cells are capable of becoming a variety of cell types or a few specific cell types depending upon in vivo or in vitro culture conditions. In other words, a cell's predetermined fate is determined by it's environment to be destined for a particular differentiation pathway such that a cell becomes one cell type instead of another cell type, for example, a stem cell's progeny cells whose “neural fate” is to become a nerve cell instead of a muscle cell or a skin cell. Typically, a cell's “fate” is irreversible except under highly specific conditions. In another example, a “CNS fate” refers to a cell capable of becoming a cell associated with the central nervous system. Conversely, a cell fated to become a neural cell can be called a “neural progenitor cell.”

As used herein, the term “neural progenitor cell” refers to a cell capable of forming a part of the nervous system, such as a nerve cell, a glial cell, etc.

As used herein, the term “neuronal subtype” refers to any cell of the neuronal system, such as a dopamine expressing neuron, a peripherin+ neuron, a motor neuron cell, etc.

As used herein, the term “cell of a neural lineage” refers to a cell that differentiated along a neural precursor pathway.

As used herein, the term “expressing” in relation to a gene or protein refers to making an mRNA or protein which can be observed using assays such as microarray assays, antibody staining assays, and the like.

As used herein, the term “differentiation” as used with respect to cells in a differentiating cell system refers to the process by which cells differentiate (change) from one cell type (e.g., a multipotent, totipotent or pluripotent differentiable cell) to another cell type such as a target differentiated cell.

As used herein, the term “cell differentiation” in reference to a pathway refers to a process by which a less specialized cell (i.e. stem cell) develops or matures (becomes more phenotypically specified) or differentiates to possess a more distinct form and/or function into a more specialized cell or differentiated cell, (i.e. neural cell, neural plate cell, pituitary cell, adrenal cell, etc.).

As used herein, the term “neural stem cell” or “NSC” refers to a cell that is capable of becoming neurons, astrocytes, oligodendrocytes, glial cells, etc., in vivo, and neuronal cell progeny and glial progeny in culture.

As used herein, the term “default” or “passive” in reference to a cell differentiation pathway refers to a pathway where a less specialized cell becomes a certain/specific differentiated cell type in culture, when not treating with certain compounds i.e. normal cell cultures conditions. In other words, a default cell results when a cell is not contacted by a molecule capable of changing the differentiated cell type (i.e. a morphogen). In contrast, “non-default” in reference to a cell refers to a differentiated cell type that results that is different from a default cell, i.e. a non-default cell is a differentiated cell type resulting from a non-default conditions.

As used herein, the term “kit” refers to any delivery system for delivering materials. In the context of cell differentiation, a kit may refer to a combination of materials for contacting stem cells, such delivery systems include systems that allow for the storage, transport, or delivery of reaction reagents (e.g., compounds, proteins, detection agents (such as CD166 antibodies), etc. in the appropriate containers (such as tubes, etc.) and/or supporting materials (e.g., buffers, written instructions for performing cell differentiation, etc.) from one location to another. For example, kits include one or more enclosures (e.g., boxes, or bags, and the like) containing the relevant reaction reagents.

As used herein, the term “inducing differentiation” in reference to a cell refers to changing the default cell type (genotype and/or phenotype) to a non-default cell type (genotype and/or phenotype). Thus “inducing differentiation in a stem cell” refers to inducing the cell to divide into progeny cells with characteristics that are different from the stem cell, such as genotype (i.e. change in gene expression as determined by genetic analysis such as a microarray) and/or phenotype (i.e. change in expression of a protein).

As used herein, the term “contacting” cells with a compound of the present inventions refers to placing the compound in a location that will allow it to touch the cell in order to produce “contacted” cells. The contacting may be accomplished using any suitable method. For example, in one embodiment, contacting is by adding the compound to a tube of cells. Contacting may also be accomplished by adding the compound to a culture of the cells.

As used herein, the term “stem cell” refers to a cell that is totipotent or pluripotent or multipotent and are capable of differentiating into one or more different cell types, such as embryonic stems cells, stem cells isolated from organs, for example, skin stem cells.

As used herein, the term “embryonic stem cell” refers to a cell of a stem cell line, such as WA-09, or a cell isolated from an embryo or placenta or umbilical cord.

As used herein, the term “adult stem cell” refers to a stem cell derived from an organism after birth.

As used herein, the term “neural cell line” refers to a cell line displaying characteristics normally associated with a neural cell.

As used herein, the term “totipotent” refers to an ability of a cell to differentiate into any type of cell in a differentiated organism, as well as a cell of extra embryonic materials, such as placenta, etc.

As used herein, the term “pluripotent” refers to a cell line capable of differentiating into any differentiated cell type.

As used herein, the term “multipotent” refers to a cell line capable of differentiating into at least two differentiated cell types.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “in vitro” refers to an artificial environment and to processes or reactions that occur within an artificial environment. In vitro environments can consist of, but are not limited to, test tubes and cell cultures.

The term “in vivo” refers to the natural environment (e.g., an animal or a cell) and to processes or reaction that occur within a natural environment.

As used herein, the term “neural plate” or “medullary plate” refers to a thickened band of ectoderm (an unpaired ventral longitudinal zone of the neural tube) in the midbody region of the developing embryo, which develops (differentiates) into the neural tube and neural crest.

As used herein, the term “progenitor” in reference to a cell or an area of cells refers to the type of cell or area of cells that would develop (differentiate into) under the appropriate conditions, i.e. when contacted with a proper growth factor, compound, extracellular signal, intracellular signal, etc. For example, “progenitor neuron” refers to a cell that has the capability to develop into a neuron.

As used herein, the term “dopamine neuron” or “dopaminergic neuron” in general refers to a cell capable of expressing dopamine. “Midbrain dopamine neurons” or “mDA” refer to presumptive dopamine expressing cells in forebrain midbrain structures and dopamine expressing cells in midbrain structures.

As used herein, the term “attached cell” refers to a cell growing in vitro wherein the cell contacts the bottom or side of the culture dish, an attached cell may contact the dish via extracellular matrix molecules and the like. As opposed to a cell in a suspension culture.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type. As used herein, the term “test compound” refers to any chemical entity, pharmaceutical, drug, and the like that were used to provide cells of the present inventions.

As used herein, the term “rosette-stage neural cell” or “R-NSC” refers to a neural stem cell type in vitro with broad differentiation potential capable of forming central nervous system (CNS) and peripheral nervous system (PNS) cells (fates) and capable of in vivo engraftment. In other words, a rosette-stage neural cell is capable of forming a rosette structure and rosette-stage neural cell populations have characteristics of neuronal differentiation.

As used herein, the term “rosette structure” or “rosette” in reference to a cell refers to a halo or spoke-wheel arrangement of cells.

As used herein, the term “increasing” in reference to a characteristic refers to a larger amount of a characteristic when compared to said characteristic in a control, such as when comparing an amount of a marker in human embryonic stems cells cultured with and without a test compound.

As used herein, the term “decreasing” in reference to a characteristic refers to a smaller amount of a characteristic when compared to said characteristic in a control, such as when comparing an amount of a marker in human embryonic stems cells cultured with and without a test compound.

The term “sample” is used in its broadest sense. In one sense it can refer to a cell or tissue. In another sense, it is meant to include a specimen or culture obtained from any source and encompass fluids, solids and tissues. Environmental samples include environmental material such as surface matter, soil, water, and industrial samples. These examples are not to be construed as limiting the sample types applicable to the present invention.

The terms “purified,” “to purify,” “purification,” “isolated,” “to isolate,” “isolation,” and grammatical equivalents thereof as used herein, refer to the reduction in the amount of at least one contaminant from a sample. For example, a cell type is purified by at least a 10%, preferably by at least 30%, more preferably by at least 50%, yet more preferably by at least 75%, and most preferably by at least 90%, reduction in the amount of undesirable cell types.

As used herein, the term “proliferation” refers to an increase in cell number.

As used herein, the term “ligand” refers to a molecule that binds to a second molecule. A particular molecule may be referred to as either, or both, a ligand and second molecule. Examples of second molecules include a receptor of the ligand, and an antibody that binds to the ligand.

The term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vivo, treatment and/or mutagenesis using for example proteins, chemicals, radiation, infection with virus, transfection with DNA sequences, such as with a morphagen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “biologically active,” refers to a molecule (e.g. peptide, nucleic acid sequence, carbohydrate molecule, organic or inorganic molecule, and the like) having structured, regulatory, and/or biochemical functions.

As used herein, the term “primary cell” is a cell that is directly obtained from a tissue (e.g. blood) or organ of an animal in the absence of culture. Typically, though not necessarily, a primary cell is capable of undergoing ten or fewer passages in vitro before senescence and/or cessation of proliferation. In contrast, a “cultured cell” is a cell that has been maintained and/or propagated in vitro for ten or more passages.

As used herein, the term “cultured cells” refer to cells that are capable of a greater number of passages in vitro before cessation of proliferation and/or senescence when compared to primary cells from the same source. Cultured cells include “cell lines” and “primary cultured cells.”

As used herein, the term “cell culture” refers to any in vitro culture of cells. Included within this term are continuous cell lines (e.g. with an immortal phenotype), primary cell cultures, finite cell lines (e.g., non-transformed cells), and any other cell population maintained in vitro, including embryos and embryonic cells.

As used herein, the term “cell line,” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

As used herein, the terms “primary cell culture,” and “primary culture,” refer to cell cultures that have been directly obtained from cells in vivo, such as from animal tissue. These cultures may be derived from adults as well as fetal tissue.

As used herein, the terms “monolayer,” “monolayer culture,” and “monolayer cell culture,” refers to a cell that has adhered to a substrate and grow as a layer that is one cell in thickness, in other words, an “attached cell.” Monolayers may be grown in any format, including but not limited to flasks, tubes, coverslips (e.g., shell vials), roller bottles, et cetera.

As used herein, the terms “feeder cell layer” or “feeder cell population” refers to a monolayer of cells used to provide attachment molecules and/or growth factors for an adjacent cell, for example, used in co-culture to maintain pluripotent stem cells. As used herein, the terms “suspension” and “suspension culture” refer to cells that survive and proliferate without being attached to a substrate. Suspension cultures are typically produced using hematopoietic cells, transformed cell lines, and cells from malignant tumors.

As used herein, the terms “culture media,” and “cell culture media,” refer to media that are suitable to support the growth of cells in vitro (i.e., cell cultures, cell lines, etc.). It is not intended that the term be limited to any particular culture medium. For example, it is intended that the definition encompass outgrowth as well as maintenance media. Indeed, it is intended that the term encompass any culture medium suitable for the growth of the cell cultures and cells of interest.

As used herein, the term “cell” refers to a single cell as well as to a population of (i.e., more than one) cells. The population may be a pure population comprising one cell type, such as a population of neuronal cells or a population of undifferentiated embryonic cells. Alternatively, the population may comprise more than one cell type, for example a mixed cell population. It is not meant to limit the number of cells in a population, for example, a mixed population of cells may comprise at least one differentiated cell. In one embodiment a mixed population may comprise at least one differentiated. In the present inventions, there is no limit on the number of cell types that a cell population may comprise.

As used herein, the term “positive cell” in relation to a stain refers to a cell that expresses a marker and thus “stains” for that marker in a detectable quantitative and/or qualitative amount above a control or comparative cell. A positive cell may also refer to a cell that stains for a molecule such as CD166.

As used herein, the term “negative cell,” refers to a cell absent detectable signal for a marker, such as a cell failing to stain following contacting with a CD166 antibody detection method.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located, adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1—Efficient Generation of Midbrain Dopa Progenitors from Human ESCs or iPSCs in 20 Days

To induce the specification of neuroepithelial cells from human pluripotent cells, the dual Nodal/BMP inhibition approach was applied for human embryonic stem cells in a monolayer culture, see Chambers et al., Nature Biotech. 27:275-280 (2009). The small molecule SB431542 represses Nodal/Activin signaling by selectively inhibiting Activin receptor-like kinase ALK4/5/7. The small molecule DMH-1 represses BMP signaling by selectively inhibiting the BMP receptor kinase ALK2. SAG (a SHH signaling agonist) was applied to induce ventral floor plate cells. CHIR99021, a WNT signaling agonist is critical to induce midbrain fate, see Xi et al. Stem Cells, 30:1655-1663 (2012). The dose of CHIR99021 requires to be optimized for every human ESC or iPSC line, mostly selected from 0.7-1.2 uM. After the induction of midbrain floor plate progenitors, FGF8b and SAG were applied to further induce mDA progenitors, see Xi et al. Stem Cells, 30:1655-1663 (2012)

1. After treated with Accutase for 5 minutes, the human ESCs or iPSCs were dissociated into single cell, and plated 2×106 cells onto a laminin-511 coated T75 flask with 15 ml E8 PSC medium.
2. On Day 2, the culture medium was replaced with neural medium (DMEM/F12, 1×N2 neural supplement, 1× Glutamax, 1 mM ascorbic acid). 2 μM DMH-1, 2 μM SB431542, 0.7 μM CHIR99021 and 1 μM SAG were added in neural medium. The culture medium was changed every other day until Day 10.
3. On Day 10, the ESCs and iPSCs are induced into midbrain floor plate progenitors, then were split into a T175 flask to further induce midbrain mDA progenitors. After treated with diluted Accutase for 5 minutes, midbrain floor plate progenitors were collected and plated onto a laminin-511 coated T175 flask with 45 ml neural medium. 0.7 μM CHIR99021 and 0.1 μM SAG were added in neural medium.
4. On Day 12, change medium with 75 ml neural medium. 20 ng/ml FGF8b and 0.1 μM SAG were added to further induce Dopa progenitors. The culture medium was changed every 3 days until Day 18.
5. On Day 18, change medium with 75 ml neural medium, 2 μM DMH-1, 2 μM SB431542, 3 μM CHIR99021, 0.1 μM SAG and 0.5 μM WNT-059 were applied to form mDA expansion medium.
6. On Day 20, the midbrain DA progenitors were generated, which can be sorted with CD166 MACS beads, or expanded into the scale needed.

### Example 2—Sorting of Pure Midbrain DA Progenitors with CD166 MACS Beads

1. On Day 20, the purity of midbrain DA progenitor were detected with CD166 (Miltenyi Biotec) and Corin antibodies (R&D systems) using standard flow cytometric analysis, which varies among different human ESC or iPSC lines. If purity >70%, the mDA progenitors can be expanded into the scale needed. If purity <70%, the mDA progenitors can be sorted with the following steps.
2. mDA progenitors were treated with diluted Accutase for 10 minutes, and then the digested single cells were collected and passed through a 40-μm strainers (1×10⁷ cells/mL).
3. The cells were suspended in PBS and FcR blocking buffer for 15 minutes, and then added anti-CD166 human polyclonal immune-magnetic beads (Thermo Fisher Scientific) for one hour.
4. After washing bead-bound cells for 3 times, the CD166-positive mDA progenitors were obtained. The purity of both CD166-positive and CD166-negative cells was detected using standard flow cytometric analysis with CD166 and Corin antibodies.

### Example 3—Expansion of Pure Midbrain mDA Progenitors

The midbrain DA progenitors can be maintained and expanded in culture for at least 5 weeks (e.g., at least 5 passages), yielding previously unobtainable numbers of mDA progenitors (on the order of producing 100 mDA progenitors from a single mDA progenitor cell).

1. On Day 20, the purity >70% mDA progenitors were plated on a laminin-511 coated T75 flask or T175 flask depend on cell numbers, 5×10⁷ cells for T75 flask and 15×10⁷ cells for T175 flask. mDA expansion medium 2 μM DMH-1, 2 μM SB431542, 3 μM CHIR99021, 0.1 μM SAG and 0.5 μM Wnt-059 were applied.
2. The mDA expansion culture medium was changed every 3-4 days.
3. mDA progenitors were passaged when confluent, mostly once a week. After treated with diluted Accutase for 5 minutes, mDA progenitors were passaged at 1:2 or 1:3 into new laminin-511 coated flasks.
4. After required scale of mDA progenitors were produced, the cells can be dissociated with diluted Accutase for 10 minutes, and frozen in CryoStor10 medium.
5. When thawed and cultured in DA neuron differentiation medium, mDA progenitors differentiated into TH+ post-mitotic neurons in 1-2 weeks.

We claim:

1. A method of purifying a population of midbrain dopamine (mDA) progenitor cells by isolating CD166 expressing cells from a differentiated population of progenitor cells to produce a purified population of mDA progenitor cells.

2. The method of claim 1, wherein the differentiated population of progenitor cells is derived from pluripotent stem cells.

3. The method of claim 1, wherein the differentiated population of progenitor cells is derived from induced pluripotent stem cells (iPSC).

4. The method of claim 1, wherein the differentiated population of progenitor cells is derived from embryonic stem cells (ESC).

5. The method of claim 1, wherein the differentiated population of progenitor cells are derived from midbrain floor plate progenitor cells.

6. The method of claim 1, wherein the differentiated population of progenitor cells comprise cells expressing one or more genes selected from EN1, PAX8, OTX2, LMX1A, FOXA2, Corin, and CD166.

7. The method of claim 1, wherein the differentiated population of progenitor cells comprise less than 70% CD166⁺ cells.

8. The method of claim 1, wherein the differentiated population of progenitor cells comprise less than 70% CD166⁺, Corin double positive cells.

9. The method of claim 1, wherein the purified population of mDA progenitor cells comprise at least 70% CD166⁺ cells.

10. The method of claim 1, further comprising expanding the purified population of mDA progenitor cells.

11. The method of claim 10, wherein expanding the cell population comprises contacting the purified population of mDA progenitor cells with a SHH agonist, a BMP inhibitor, a Nodal/Activin inhibitor, a GSK3 inhibitor and a PORCN inhibitor for period of time until a sufficient number of mDA progenitor cells are produced.

12. The method of claim 11, wherein the expansion does not alter the phenotype of the purified population of mDA cells.

13. A method of expanding a population of midbrain (mDA) cells, wherein the method does not alter the phenotype of the mDA cells, comprising providing a population of mDA cells and contacting the culture with a SHH agonist, a BMP inhibitor, a Nodal/Activin inhibitor, a GSK3 inhibitor and a PORCN inhibitor for period of time until a sufficient number of mDA progenitor cells are produced.

14. The method of claim 12, wherein prior to expansion the population of mDA cells are at least 70% CD166⁺.

15. A method of expanding a population of midbrain (mDA) cells, wherein the method does not alter the phenotype of the mDA cells comprising:
a. isolating CD166 expressing cells from the population mDA cells to provide a purified population of mDA cells; and
b. culturing the purified population of mDA cells in the presence of a SHH agonist, a BMP inhibitor, a Nodal/Activin inhibitor, a GSK3 inhibitor and a PORCN inhibitor for period of time until a sufficient number of mDA progenitor cells are produced thereby expanding the population of mDA cells.

16. The method of claim 15, wherein the purified population of mDA progenitor cells comprise at least 70% CD166⁺ cells.

17. A method of expanding a population of midbrain (mDA) cells, wherein the method does not alter the phenotype of the mDA cells comprising:
a. providing a population of at least 80% CD166⁺ mDA cells; and
b. culturing the CD166⁺ mDA cells in the presence of a SHH agonist, a BMP inhibitor, a Nodal/Activin inhibitor, a GSK3 inhibitor and a PORCN inhibitor for period of time until a sufficient number of mDA progenitor cells are produced thereby expanding the population of mDA cells.

18. The method of claim 11, wherein the period of time is about between 1-5 weeks.

19. The method of claim 13, wherein the sufficient number of mDA progenitor cells is at least 10⁹ cells.

20. The method of claim 11, wherein the BMP inhibitor is DMH1.

21. The method of claim 20, wherein the concentration of DMH1 is between about 0.1 μM to about 10 μM.

22. The method of claim 20, wherein the concentration of DMH1 is between about 1 μM to about 5 μM.

23. The method of claim 20, wherein the concentration of DMH1 is about 2 μM.

24. The method of claim 11, wherein the GSK3 inhibitor is CHIR99021.

25. The method of claim 24, wherein the concentration of CHIR99021 is between about 0.1 μM and about 5 μM.

26. The method of claim 24, wherein the concentration of CHIR99021 is between about 1 μM and about 5 μM.

27. The method of claim 24, wherein the concentration of CHIR99021 is about 3 μM.

28. The method of claim 11, wherein the SHH agonist is SAG.

29. The method of claim 28, wherein SAG is at a concentration of between about 0.01 μM and 5 μM.

30. The method of claim 28, wherein the SHH agonist is SAG and the concentration of SAG is between about 0.05 μM and 1 μM.

31. The method of claim 28, wherein the concentration of SAG is about 0.1 μM.

32. The method of claim 11, wherein the Nodal/Activin inhibitor is SB431542.

33. The method of claim 32, wherein the SB431542 is at a concentration of between about 0.1 μM and about 10 μM.

34. The method of claim 32, wherein the SB431542 is at a concentration of about 2 μM.

35. The method of claim 11, wherein the PORCN inhibitor is Wnt-059.

36. The method of claim 35, wherein the concentration of Wnt-059 is between about 0.1 μM and about 1 μM.

37. The method of claim 35, wherein the concentration of Wnt-059 is about 0.5 μM.

38. The method of claim 1, wherein the CD166 expressing cells are isolated by FACS or MACS.

39. An in vitro method of producing a midbrain dopamine (mDA) progenitor cell population comprising:
a. culturing a population of less than 5 million stem cells for a first period of time such that small cell clusters are formed to produce a first cell population;
b. contacting the first cell culture with the BMP inhibitor, the GSK3 inhibitor, the SHH agonist, and a Nodal/Activin inhibitor for about seven to twelve (7-12) consecutive days to produce a first midbrain floor plate progenitor cell population;
c. passaging the midbrain floor plate progenitor cell population to produce a passaged cell population;
d. contacting the passaged cell population with the GSK3 inhibitor and the SHH agonist for about two (2) days to produce a second midbrain floor plate progenitor cell population;
e. contacting the second midbrain floor plate progenitor cell population with FGF8b and the SHH agonist for about six (6) consecutive days thereby producing a mDA progenitor population;
f. contacting the mDA progenitor cell culture with the SHH agonist, the BMP inhibitor 1, the Nodal/Activin inhibitor, the GSK3 inhibitor and a PORCN inhibitor for about two (2) days to produce an expanded mDA progenitor cell population;
g. purifying the mDA progenitor cell population by isolating CD166 expressing cells from the cell population to produce a purified mDA progenitor cell population; and
h. expanding the purified mDA progenitor cell population by contacting the culture with a SHH agonist, a BMP inhibitor, a Nodal/Activin inhibitor, a GSK3 inhibitor and a PORCN inhibitor for a second period of time until a sufficient number of mDA progenitor cells are produced.

40. The method of claim 39, wherein the second period of time is between about 7-12 days.

41. The method of claim 39, wherein the population of stem cells is less than 1 million cells.

42. The method of claim 39, wherein the population of stem cells is less than 500,000 cells.

43. The method of claim 39, wherein the sufficient number of mDA progenitor cells is at least 10⁶ cells.

44. The method of claim 39, wherein the sufficient number of mDA progenitor cells is at least 10⁷ cells.

45. The method of claim 39, wherein the sufficient number of mDA progenitor cells is at least 10⁸ cells.

46. The method of claim 39, wherein the first period of time is about 2 days.

47. The method of claim 39, wherein the BMP inhibitor is DMH1.

48. The method of claim 47, wherein the concentration of DMH1 is between about 0.1 μM to about 10 μM.

49. The method of claim 47, wherein the concentration of DMH1 is between about 1 μM to about 5 μM.

50. The method of claim 47, wherein the concentration of DMH1 is about 2 μM.

51. The method of claim 39, wherein the GSK3 inhibitor is CHIR99021.

52. The method of claim 51, wherein the concentration of CHIR99021 is between about 0.1 μM and about 5 μM.

53. The method of claim 39, wherein the GSK3 inhibitor is CHIR99021 and the concentration of CHIR99021 in step (b) is between about 0.7 μM and about 1.2 μM.

54. The method of claim 39, wherein the GSK3 inhibitor is CHIR99021 and the concentration of CHIR99021 in step (d), (f) and (h) is between about 1.0 μM and about 5 μM.

55. The method of claim 54, the concentration of CHIR99021 is about 3 μM.

56. The method of claim 39, wherein the SHH agonist is SAG.

57. The method of claim 56, wherein SAG is at a concentration of between about 0.02 μM and 5 μM.

58. The method of claim 56, wherein the SHH agonist is SAG and the concentration of SAG in step (e) is between about 0.1 μM and 2 μM.

59. The method of claim 56, wherein the concentration of SAG is about 1 μM.

60. The method of claim 39, wherein the SHH agonist is SAG and the concentration of SAG in step (0 and (h) is between about 0.02 μM and about 1 μM.

61. The method of claim 60, wherein SAG is at a concentration of between about 0.05 μM and about 0.5 μM.

62. The method of claim 60, wherein SAG is at a concentration of about 0.1 μM.

63. The method of claim 39, wherein the Nodal/Activin inhibitor is SB431542.

64. The method of claim 63, wherein the SB431542 is at a concentration of between about 0.5 μM and about 5 μM.

65. The method of claim 63, wherein the SB431542 is at a concentration of about 2 μM.

66. The method of claim 39, wherein the PORCN inhibitor is a porcupine (PORCN) inhibitor.

67. The method of claim 66, wherein the PORCN inhibitor is Wnt-059.

68. The method of claim 66, wherein the concentration of Wnt-059 is between about 0.1 μM and about 1 μM.

69. The method of claim 66, wherein the concentration of Wnt-059 is about 0.5 μM.

70. The method of claim 39, wherein the FGF8b in step (e) is at a concentration of between about 5 ng/ml and about 50 ng/ml.

71. The method of claim 70, wherein the FGF8b is at a concentration of about 20 ng/ml.

72. The method of claim 39, wherein the CD166 expressing cells are isolated by FACS or MACS.
